(19) United States
(12) Patent Application Publication
(10) Pub. No.: US 2011/0203597 A1
(43) Pub. Date: Aug. 25, 2011

(54) SIDE-SPECIFIC FLUID-RETAINING CONDOM SYSTEM

(76) Inventors: Leon B. KASSMAN, Rockville Center, NY (US); Stewart Swiss, Edgewood, NY (US); Harry Herz, Edgewood, NY (US)

(21) Appl. No.: 13/031,787

(22) Filed: Feb. 22, 2011

Related U.S. Application Data
(60) Provisional application No. 61/338,421, filed on Feb. 19, 2010.
(60) Provisional application No. 61/342,815, filed on Apr. 20, 2010.
(60) Provisional application No. 61/404,865, filed on Oct. 12, 2010.

(51) Int. Cl. A61F 6/04

(52) U.S. Cl. 128/844

(57) ABSTRACT

A condom having an elongated shape with an inner and an outer surface is provided. A barrier or strut is formed on the condom which prevents any liquid exchange between the inner and outer surfaces during the use of the condom.

### RELATED APPLICATIONS

This application claims priority to the following provisional applications: Ser. No. 61/338,421 filed Feb. 19, 2010; Ser. No. 61/342,815 filed Apr. 20, 2010 and , Ser. No. 61/404.865 filed Oct. 12, 2010 all incorporated herein by reference

### BACKGROUND OF THE INVENTION

A. Field of Invention

This invention relates generally to methods of contraception and prophylaxis against diseases transmittable by sexual contact. More particularly, the invention concerns a new condom system consisting of several distinguishable novel improvements of a conventional male or female condom, including providing several novel means of providing a barrier between the inner and outer surfaces or sides of the condoms. All improvements were created in order solve the problems created by conceptualizing condoms from a perspective different from the way they have been previously conceived.

B. Description of the Prior Art

A condom (typically made from a non-porous material, including synthetic rubbers, latex, polymers, co-polymers and sheepskin) is a commonly used and generally effective barrier method used by either partner to provide complete isolation against transfer of bodily fluids from one partner to the other. Such devices are effective for prevention of transmission of STDs and for contraception; however the physical barrier must remain intact. In some cases however, the condom or the like may develop microscopic leaks or may contain small and perhaps unnoticeable perforations which can permit the transfer of microorganisms or of sperm across the barrier. In other cases the condom may rupture and permit substantial transfer of bodily fluids with the consequence of possible infection or conception. Although a complete rupture of a condom is relatively rare, it has been found that leaks sufficient to permit the escape of microorganisms in infectious amounts are by no means uncommon. In fact, the AQL (quality limit) posted by the FDA is 0.04 meaning that due to the imperfections known to exist in all latex condom manufacture, a very few of each lot that actually passed all FDA sampling testing is acknowledged by them that they will fail during regular use. Accordingly, the recent emergence of more serious and even fatal STDs such as AIDS has caused concern that even relatively reliable total barrier methods may not be satisfactory. Therefore, included in the preferred embodiment of present invention is a unique means to provide a second, chemical, barrier or treatment behind the barrier of the condom, on the male side of the condom, where the chemical has been demonstrated to kill nearly all pathogens, on contact, including HIV/AIDS.

In the past condoms have been thought as a product in and of itself whose purpose was to be donned onto the penis to serve as a barrier against the transmission of diseases during vaginal, anal, or oral sexual encounters to, or from either partner. Similarly, condoms have been used on various inanimate devices and objects to add a measure of safety and cleanliness when the devices are used for stimulation. However existing condoms are not satisfactory for several reasons.

A specific need exists in as much as the preponderance of the sexual transmission of disease with percentages that may exceed 60% are in the direction from males, infecting, then non-infected females, or even when the male may inadvertently re-infect and already infected female, and while condoms alone serve as the only known means of stopping the spread of these life threatening and certainly life altering diseases, and also serve as an excellent form of contraception, and since condoms break, or tear, or come off during sex, or may be removed by men at one time or another during sexual activities, it would be best, considering the above, if chemicals that could kill ambient viruses in semen or pre-ejaculate and de-activate sperm and/or remain on the surface of the males penis or in any folds there-on, therefore available to dose the penis but be restricted from the full and complete dosing of the interior of the vagina.

### SUMMARY OF THE INVENTION

For the purposes of the present invention, a typical condom consists of a separate inner surface (part actually in contact with the penis), an outer surface (part that comes into contact with vaginal, oral, or anal surfaces), closed-end (part that acts as a repository of ejaculatory fluids) and bead-end (part containing the factory intra-rolled portion used to roll down and cover the shaft of the penis). The inner surface is hereafter called the male-side, and the outer surface is hereafter called the female-side of the condom.

The reason for distinguishing between parts of the condom becomes necessary in order to understand how the invention is actually a new condom system of interacting modifications that are required so as to achieve the various specific objectives of the invention. One objective is to isolate the male-side from the female-side during use and to maintain treatments (fluids or powders) that are introduced to the male-side, using a number of different means, from migrating from that side during manufacture, storage, or use. Similarly, the system enables the isolation of treatments to the female-side from similar migration or leakage.

The purpose of the treatments is to augment and extend the effects of the barrier provided by the latex or other elastomeric film itself. An alternative method for preventing the transmission of sexually transmitted diseases is to kill or inhibit the pathogenic microorganisms that may be in semen and vaginal secretions so that they are incapable of infecting the partner's tissues, thus causing disease. While intra-vaginally placed spermicides have been used for contraception, alone or in combination with barrier methods, antimicrobial materials have not been approved for said use to prevent STDs. This may be because many of such materials have been shown to irritate internal tissues. Some prior art references, such as, Howett et. al. (U.S. Pat. Nos. 6,192,887, 6,458,346, 6,635,242) and Bergeron et.al. (U.S. Pat. No. 7,192,607) have demonstrated the effectiveness in both laboratory and actual clinical trials of the identical compounds differentially called sodium dodecyl sulfate (SDS) and sodium lauryl sulfate (SLS). The FDA, along with most chemists consider both names to be an accepted manner of expressing the same number (25) of carbon atoms in the compounds chemical structure. It is important to make note that SDS is one of the most often used chemical ingredients used regularly and commonly by man and, indeed, the compound has been on the FDA's GRAS (Generally Recognized As Safe) listing of chemicals automatically considered safe for humans to use. The present invention uses this chemical as one active ingredient that can function as a microbicide, although by no means is the invention restricted to being used solely for that chemical.

The art is rich with inventions that focus on generating pleasure for women by introducing nubs, ridges, lines and extra material onto the condom body with the additional an concomitant purposes of reducing slippage and leakage. In addition to one product called Inspiral™, the inventions discussed in U.S. Pat. No. 6,074,671 (Oldham), U.S. Pat. No. 6,298,852 (Michelson), U.S. Pat. No. 6,983,751 (Osterberg), U.S. Pat. No. 5,954,054 (Johnson), U.S. Pat. No. 6,135,112 (Strauss), and U.S. Pat. No. 5,715,839 (Strauss), WO 97/30668, Design Pat. No. 420,127, U.S. Pat. No. 6,308,708, Design Pat. No. 252,949, Design Pat. No. 448,471 are particularly noteworthy for background. However, the present invention provides novel improvements not foreseen by these earlier inventions.

To achieve and maintain the treatments to the separate sides, the treatment itself is introduced either directly at the factory to the condom itself, or maybe introduced just before use by means of special applicators that are packaged along with the condoms. To maintain the treatments post their introduction to the condom, regardless of when and how they are introduced, certain structural changes in the part of the condom ordinarily covering the shaft of the penis are part of the invention. These structural changes not only retain the treatments but act simultaneously to maintain erectile turgidity by restraining the normal outflow of ambient fluids that filled cavernosal chambers causing the erection while stimulating internal and external vaginal surfaces in a manner that cause additional excitement.

A further feature of the invention is a unique and novel condom-system, which in various iterations may include a reconfigured condom, a detachably attached plug that fits within the bead-end, at least one or several thickened circumferential rings of same material at or toward the condom bead-end and which extend both inward and outward at that point to constrict (male) and stimulate (female) anatomical parts, and one or more types of applicators used variously to orient the condom and hold and/or provide a means of treating the male-side of the condom with any fluid/powder and to also act to hold such treatment from migrating to the opposite side of the condom during manufacture, storage, or use.

This patent application further extends Kassman's Drug Delivery Package U.S. Pat. No. 7,617,827 that was awarded on Nov. 17, 2009. That Patent substantially covers a means of adding a medicinal substance, particularly to the outside of a conventionally fabricated condom, made from any elastomeric product, by using a specially constructed/pre-packaged applicator at a time very close in proximity to the time the condom is actually being used, and keeping that medicinal substance in a pouch or container that maintains the substance out of direct contact of the condom until the time just before use.

The literature has provided for devices, used for other purposes, to be attached to or detachably-attached to condoms. Schroeder U.S. Pat. No. 4,726,359 described ‘end-caps on rolled condoms that were meant to indicate the direction of unrolling the condom and to maintain sterility. Since these caps fit over the whole condom and are not, as the present patent maintains, disks inserted into the condom to prevent migration of fluids, this patent is not persuasive over the present application. Foldesy U.S. Pat. No. 5,163,448 mentions the use of a ‘rolled-ring’ in connection with the application of a medicament to a condom. The present invention is round but not at all a ring, but, instead, a solid round disk, And, it is not used to disperse lubricants but, instead, to hold any previously dispersed lubricant or microbicide in the same position that it was originally dispersed into. Also described is a ‘plug’. Here, too, the described plug acts as a reservoir, a container, holding a medicament or other material but, in no way serving as a block against migration of any lubricant. Jones U.S. Pat. No. 5,316,019 describes a ring member and a tolled-up condom but the ring is exactly that, a ring, used as a means of applying the condom with said ring being frangible therefore allowing it to expand as it is drawn over a penis diameter presumably larger than the diameter of the ring at rest. From the description, the ring and the condom are fully immersed in the included lubricant and therefore the ring does not serve as a means of preventing lubricant migration. Kuyumciyan U.S. Pat. No. 5,471,998 and Steer U.S. Pat. No. 5,531,725 describe tubular applicator devices, one a hollow tube and the other a strip of adhesive tape with the purpose of improving condom application or condom retention on the penis but neither device mentions preventing lubricants from migrating from one part of the condom to another.

The current patent application focuses primarily on the inside surface (male-side) of the male condom and by extension, will also apply to the male-side surfaces which exist opposite from the interior (female-side) surfaces of a female condom once it is inserted into the vagina.

The current patent shall cover natural rubber latex, polyethylene, polyisoprene and any other polymer which may or may not have elastomeric properties permitting even the most limited amount of stretch, or to compositions and configurations which may be constructed of any substance (of less or no elastomeric properties) so as to form a tubular shaped configuration roughly open at one end and closed of the same, where the total device is made of a single substance, or of other substances that can be bound to the substance used to form the preponderance of the device. The device may be used for such purposes as contraception, prevention from disease, support, or the means of dispersal of any medicinal product directly to the penis and/or to the vagina.

Condoms, predominantly male condoms, but female condoms as well, have been designed largely to conform to the roughly tubular or somewhat triangular configuration (see Kassman patent application Ser. No. 10/016,219 Hermaphroditic Condoms) and fabricated from a substance that will, or configured in a manner to essentially fit themselves, post donning, to the physiological extent or confines of either an erect penis or to largely expand toward and to the walls of the vagina.

To accommodate human anatomic differences these devices have be made from mostly some kind of substance that not only has elastomeric properties, permitting some form of stretch, but also of substrates that are significantly impervious to the passage of sperm and similarly impervious to the passage of many or most bacteria, viruses, fungi, or other pathogens that cause almost any type of Sexually Transmitted Infections (STIs), as well as the transmission of those infections mostly associated and which mostly originate in women's vagina like vaginitis. The current patent application's extent is meant to include sheaths of all types designed to prevent the transmission of any unwanted, naturally occurring, or other condition whether it be infectious to the opposite partner or not.

Associated with the development of protective sheaths (regardless of whether they are male or female condoms or condom like structures), has been a parallel development or use of naturally occurring products or combinations of products whose lubricious nature could be used to more easily and comfortably allow an erect male penis to be inserted into a vagina. Although many women's vagina's have natural means where substances are emitted that act to lubricate the internal tissues, some women do not, and there are instances where such lubrication is impossible, truncated, or non-existent. To overcome these circumstances men and women have been using many different substances and methods to provide more comfort during intromission. These substances have included bees wax, lard, butter, grease, olive oil, whale oil, and any other substance that offers some slippery-ness during use including saliva.

Eventually, through advances in technology, substances were developed that were highly lubricious, maintaining that characteristic for years (when sealed an-aerobically in plastic or foil condom packages) so that condoms could be used with more comfort. Allied to this development was the discovery that all petroleum based oil products, and many naturally based oil products, were not recommended for use—especially never with natural rubber latex substrate products) because, over time, and relatively quickly, the petroleum oil acts to interrupt the natural rubber latex chemical bonds with the net result of weakening the surface therefore compromising the ability of the latex to serve as a barrier to any pathogens.

When water based lubricants and those that used silicone as a medium were discovered and implemented, the problem concerning disintegration of the latex bonds was ended and, today, all lubricants used in the natural rubber latex products are either water-based lubricants or silicone based lubricants—as now required by FDA statute. Water is also an excellent medium to dissolve many ingredients. Therefore, the current patent envisions the use of a water based lubricant on the female-side of the invention and, additionally, the use of water but not as a lubricant to be the medium into which a microbicide or spermicide is dissolved and then variously introduced to the male-side of the invention either in the factory or by virtue of an applicator or a frangible pouch shortly before or during the actual donning of the condom and before actual use during any form of sexual contact between individuals or between an individual and a device used for stimulation (masturbatory use).

Other elastomeric products, but not fabricated from natural rubber latex, do not share that chemical deterioration problem, and, therefore, there is no proscription about the use of petroleum or natural based oils for them, except where the manufactures of those oils indicate that they should not be used openly on the skin and particularly not on parts of the skin where those tissues will be adversely affected.

One characteristic of silicone lubricants is that they tend to spread from molecule to molecule permeating across almost all non-absorptive surfaces by means of capillary action. This characteristic is used in the manufacture of all conventionally sealed latex condoms. Drops of the lubricant are added immediately after the time the already inter-rolled condom drops on to a foil laminate band and prior to the other half of the laminate is joined and sealed and then crimped and cut into the roughly 2″×2″ squares commonly available and seen as conventional condom packages.

Ordinarily, the position of the condom that dropped onto the foil is not routinely controlled and therefore when sitting on the foil waiting for the drops of lubricant, they may be oriented with their closed end up or, just as commonly, oriented with their open end up. Therefore, the lubricant may drop into the interior portion (male side) of the condom or it may drop on the exterior portion (female side) of the condom. Manufactures rely on the natural capillary action of the lubricant to spread itself, even throughout the inter-rolled portion of the condom, over time. This is the conventional method used in the industry and to make certain that the correct amount of lubricant with sufficiently limited viscosity was used, so that the lubricant will ‘flow’ into all condom recesses and folds. The FDA requires that a certain percentage of the lubricant should be spread over the entire surface of the total condom (interior and exterior/male side and female side) and regularly tests to make certain that this condition applies.

The spread of the lubricant on all surfaces of the condom the lubricity of the lubricant is a concern because manufacturers do not want the condom so slippery as to easily or immediately allow the condom to be dislodged off the penis during coitus. The present invention strictly controls the surfaces treated and migration of the lubricant.

With the exception of the Frank Sadlow (U.S. Pat. No. 5,857,466) patented ‘multi-size’ condoms, most condoms are made on standard dipping mandrels and are of a uniform width (diameter) particularly for the part of the condom commonly used to cover the shaft of the penis. Thus, measuring form the open (bead) end of the condom the first 3″ to 5″ of the condom is configured to compressively hold itself around the erect penis width sufficient foot pounds of compressive force to overcome the effects of the lubricant. In fact, many condoms are designed so that they begin to flare out around the glans of the penis starting approximately 4″ to 5″ from the same bead (open) end with such flare ending in the reservoir tip that would ordinarily be positioned over the tip of the glans end of the penis. Therefore, in this area, the lubricant, that had deposited itself there serves to make the folds of the extra, flared, portion of the condom, during coitus, more easily move over the glans of the penis therefore achieving the designed for additional frictional sensation and, hoped for, satisfaction from using that ‘expanded’ form of condom product.

Although there are variations of this, as packaged, by different manufactures, essentially, this is the state of affairs currently. If an individual wants to add lubrication, whether to the inside or the outside of the condom, in addition to the ambient lubrication already included, there are a myriad of water-based or silicone-based products, consistent with the chemical composition of the natural rubber latex, that are available and which are commonly used.

It is an object of the present invention to provide a male condom that is designed and constructed to be highly effective in preventing unwanted pregnancy as well as combating public health concerns, such as the transmission of STDs through the exchange of bodily fluids during sexual activity.

The resulting condom system has several advantages such as:

1) To provide a male condom that is easy to properly apply to the penis.

2) To provide a male condom that is securely retained on the wearer's penis during sexual activity.

3) To provide a male condom including a microbicide/spermicide or other medicinal chemical.

4) To apply the microbicide/spermicide medicinal chemical to the interior (male side) of the condom and to retain it there during coitus.

5) To automatically (by virtue of the act of donning) disperse the microbicide/spermicide from a reservoir contained within the condom or within an applicator attached to the condom at the time of use.

6) To introduce said microbicide/spermicide at the time of the manufacture or by the user at the time of use, in a manner so as to maintain said chemical on the male side of the condom using either physical/mechanical and/or physical/mechanical/chemical barriers and to delimit its migration to the female side of the condom and to delimit its migrating into the vagina during coitus.

7) That, notwithstanding the uniformly rounded, cross-section profile of formed condom rings as described by Strauss in U.S. Pat. No. 5,715,839, the present invention specifically forms thick rings of highly articulated shape that are designed to retain the chemical microbicide/spermicide on the male side of the condom. The added thickness of the latex has the effect of adding compressive pressure to the penis resulting in retarding the outflow of ambient fluids that filled cavernosal sacks within the penis thereby contributing to the erection. By retarding outflow the thicker circumferential rings result in erectile maintenance. Furthermore, concurrently due their shape and thickness, and by virtue of specific added design, the reciprocal part of these same rings on the outside (female-side) of the condom shall act to further excited female anatomical parts that they may contact during coitus.

The information contained within U.S. Pat. No. 6,135,112 is illustrative of the current state of the art. it fails to provide for erectile maintenance preferring to advise against tightness as causing discomfort. That patent contains general language concerning slippage and leakage and similar language regarding clitoral stimulation. Other than the minimum number of rings and their relative spacing the patent is inadequate in foreseeing that fluids on the male side of a condom must be blocked by engineered structures not by simply arraying a number of similar sized dykes that are quickly and easily over-topped.

8) New System Condoms will not have the tendency to slip-off during coitus because lubricants are restricted to the female-side of the condom.

9) Female anatomical surfaces that may be irritated by certain spermicide or microbicides will be protected from exposure because said chemicals are retained on the male-side of the condom.

The new condom system offers a variety of way of treating the male-side of the condom with microbicides/spermicides.

One of the problems with microbicides is that they contribute/cause toxicity in the vagina, along with abrasive changes in vaginal epithelial tissues. These changes potentially weaken the normal blocking power of skin type tissue (even internal lining are a kind of skin) by causing an topical abrasion—resulting in an easier pathway for infection than if the microbicide were not used. Some abrasions have been seen colposcopically during clinical trials.

Since treatments applied to the inside of the condom will, over time, migrate by capillary action to the outside of the condom the New Condom System isolates the microbicide to the male-side of the condom. This is accomplished by inserting a detachably attached plug into the bead end of the condom after the condom has been dosed during packaging. The plug consists of a roughly spherical structure which may or may not be solid or may or may not be a ring with its center void. At the periphery of the plug a concave groove is created to hold the finished/bead-end of any rolled condom, The plug is constructed of an elastomer plastic of sufficient rigidity to overcome the compressive forces of the attached condom without deforming. The dimensions of the plug may vary in accordance with the circumference of the condom finished/bead-end with the preferred embodiment being at least 5% greater in circumference than the attached condom so that the condom will hold onto the plug by compressive force. This dimension may be increased which will directly increase the compressive force so as to improve the grip of the condom and resist its dislodging prematurely. Additionally, such additional compressive force will further prevent any treatment introduced into the male-side of the condom from migrating or leaking out during manufacture, shipment, or shelf-storage. The grooved part of the plug is also referred to as its rim. The plug is designed with a finger hold central strut to permit it to be easily grasped and removed. Additionally, the top most edges of the plug are diminished in two areas further enabling the plug to be easily removed from the condom just prior to donning. The fact that the plug sits in the rolled up section of the condom orients the user as to the correct-way-on donning direction to achieve additional speed in donning as well as to assure that the condom is donned in the correct-way-on orientation so that it may be fully rolled onto the erection.

In another preferred embodiment the plug's center is removed leaving mainly the grooved rim to which a condom can be attached and held. In this iteration, the structure of the rim is increased in rigidity to compensate for the loss of its center strut. Such increase may be accomplished by changing the formulation to a more rigid form of plastic ingredient or by increasing the wall thickness of said substrate. The rim is further bifurcated (split into an upper section and a lower section) to which the condom is actually assembled. The bifurcated halves are further modified with screw threads enabling each half to be mated together with at least ½ turn. Further changes to the top half of the rim causes a partition designed to fit against the outer side of the finished/bead-end of the condom once it is attached in a manner that further compresses the finished/bead-end so as to provide a further barrier against the migration or leakage of any treatment that was introduced into the male side of the condom after the condom was assembled to rim. The rim may be used as a round applicator to aid in the positioning and donning of the attached condom. Once dosed, the top of the applicator is screwed down over the bottom half (to which the condom has been pre-factory applied) applicator in a manner so that an interior stop on the top cap presses against the side of the condom preventing any of the microbicide from leaking out during shipping or shelf storage. This is the same method used to prevent liquids from spilling out from plastic bottles if the bottle were inverted. Prior to use the user unscrews the top half and then uses the bottom half to position and then lower the condom (with its contents held within) over the erection. This action dons the condom on to the penis with the final transfer being accomplished using the edge of the thumb to remove the condom from the applicator. Post application the applicator is discarded.

Another iteration uses a oblong applicator onto which a condom is factory mounted and into which, post such mounting, an amount of microbicide/spermicide is treated. The applicator is similar to the applicator described in Kassman U.S. Pat. No. 7,617,827 except that the top lip of the applicator was modified to better hold a snap cap made to fit over and snap down onto a condom that had been applied to the applicator. A snap-on cap is used to hold the microbicide fluid within the condom until the condom is used. The applicator is then used to squeeze open the condom, center it over the erection, and then to lower it over the erection. By thumb rolling the condom off the applicator it is then transferred to the penis and the applicator is discarded. Another iteration positions a frangible pouch/pellet tack-attached to the interior wall of the condom and further positions it below the internal rolled-up bead so as to roughly fall on the second two inches of the shaft of an erect penis or to start 2 inches from the point that the pouch/pellet begins measured from the fully unrolled finished/bead-end and extending for 1 additional inch toward the reservoir tip of the condom. The width of the pouch/pellet will be at least ¼ inch and its height above the wall of the condom to which it is attached will be at least ¼ inch. The pouch/pelled will be manufactured of a frangible material compatible with the condom substrate and which may be treated in a manner that will assure that its contents will be disgorged when the penis is inserted and the condom rolled down. The actual fracturing would be caused by rolling it down. To assist in this function the pouch/pellet may be pre-perforated.

Alternatively, the wall of the elastic condom, formed via dipping or heat sealing could be initially formed in a fashion so that a small self-material bladder is formed on the outside of the condom (which becomes the inside of the condom when it is striped off the mandrel. This bladder may or may not be filled with air and it may or may not be totally separated space from the wall of the condom on which it was formed. A secondary step would be needed to fill the bladder with fluid, seal it closed if were not already thus sealed and further create, as necessary, such weak points in the bladder's wall so that it easily ruptured either as the condom, then attached to the applicator were squeezed open or when the male's penis was thrust into the condom during donning.

Another iteration features a conventional condom configured on an applicator with a frangible pouch/pellet suspended just above the applicator in such a manner that when the apparatus is lowered onto the erection the penis itself may fracture the pellet . The pouch/pellet is permanently adhered to the applicator (as in the illustration) attached at the lateral sides of the applicator. When the applicator is squeezed open the lateral sides move in opposite direction causing the pre-perforated frangible pouch/pellet to fracture thereby disgorging the contents that were in the pouch/pellet into the male-side of the condom detachable-attached to the applicator below. The act of squeezing open the applicator in a manner during the moment that the applicator is being used to don the condom causes the pouch/pellet to be fractured thus releasing its microbicide/spermicide contents onto the penis. The action of inserting the penis into the condom using the applicator further drives and spreads the medicinal material from the then fractured pouch toward the business (reservoir-tip) end where it would best available to kill viruses.

The actual pouch/pellet would be of a very soft medical grade material—much softer and more fragile than a condom itself. The material used to hold the pouch/pellet in place would be compatible with the applicator wall to which it becomes applied. A cyanoacrilitic or a rubber cement, or any other material commonly used to hold one type of elastomer to another without damaging the walls of either would be used.

For the iteration where the frangible pouch is actually attached to the wall of the condom, the material used to attach said pouch, and the number and configuration of the points at which such pouch is attached may be different with there being at least one such attached point. Similarly, the number and places as well as the means of attaching a pouch configured to be attached circumferentially to the inside of the condom may vary.

The structure of the frangible pouches may also vary in terms of their shape, the positioning of the frangible elements, the number and shape of the frangible areas or lines of frangibility may also differ. The position of the frangible lines may or may not influence the direction of the flow of the contents of the packages.

The material within any of the frangible pouches may be a spermicide, Or the material may be a microbicide. Or the material may be a substance which helps the condom better stick to the penis regardless of whether the penis was then wet or dry.

The material within any of the frangible pouches may stimulate the penis to achieve a greater erection. Or the material may be such to sustain the length of time that the naturally occurring erection is maintained. Or the material may decrease the sensitivity of the skin of the penis during initial or further friction particularly if the material was time reactive.

Alternatively, in the Condom Applicator model, the frangible pouch (possibly even thin coated paper that was perforated in advance, and then with another very thin coating externally applied post perforation to retain the medicinal product. That pouch could be attached to the sides of the applicator prior to the condom being also attached, so that it positioned itself, depending on the length of the same material loops that would be fitted over the applicator side holding areas, just within the inside of the then being attached condom.

The spreading of fluids is a necessary part of the new condom system. This is different from the initial dispersal. In dispersal, a fluid held within the pellet/pouch would, post rupture of the pouch membrane issue out from the pouch and mostly flow with gravity (therefore, momentarily, at least, toward the bead end of the condom but still on the inner side surface of the condom). This fluid may also be dried and be in the form of an extremely fine powder that would instantly mix with any ambient liquid/lubricant or with pre-ejaculate and with semen. If in liquid form, the flow outside over the open end of the condom, would almost immediately be impeded by the presence of the penis that either immediately post rupture is thrust into the condom as it is donned thru forming a kind of piston pushing the probably still gushing liquid toward the reservoir tip end and/or at least along the inside surface of the condom. Or, should the opening squeezing torque exerted on the applicator and transferred, at least, in part, depending upon the position of the pouch (if glued to the interior condom wall) or on the position of the pouch suspended and hanging freely between the interior sides of the condom and held in place by loops of same pouch material that had been detachably attached to the side indentions formed originally as part of the applicator, were NOT sufficient to fully fracture the frangible pouch, then certainly, the penis itself, as it it thrust into the condom would fracture the pouch membrane and immediately drive the then disgorging liquid or gel toward the reservoir tip end.

The goal of the above preferred iterations was to introduce a treatment into the male-side of the condom and retain it there. To help accomplish the patent further envisions that certain modification may be required to the condom itself. In addition to the iterations above, the bead end of the condom may be altered. The purpose of this alteration is an additional measure to further retard the migration of fluid treatment that was in the reservoir end and to further retard leakage of any fluid from the male-side.

A series of at least one internally (toward the penis) facing struts can be engineered to the dipping mandrel . These circumferential struts, cause by either engraving shaped, specially configured, indentations and/or extensions on mandrel formers, so that when dipped the latex adhered and was formed into one of more circumferential struts. These struts may have single or multiple tiers, they may be rounded or comma shaped, and may be in any configuration that will enable the vulcanized rubber or elastomer substrate, once fully molded to be detached from the former. Additionally, the struts may be of a width and height of at least 1/16 inch and they may be uniformly shaped or have molded extensions to their peripheral configuration to that such extensions may protrude further in parts of the extension than at other parts of the extension. On example of the shape of the extension would be to create a continuous rib or ridge shaped like the aft part of a airplane wing . To allow for penises of different diameters, the thickness (width) of any two such ribs or the amount that they protrude from the condom wall may be different.

### BRIEF DESCRIPTION OF THE DRAWINGS

FIG. 1—Detachably-Attached Plug that can be used for mounting a condom in accordance with this invention;

FIG. 2—Condom detachably-attached to the plug of FIG. 1;

FIG. 3—Bifurcated Plug with top acting as compressive cap;

FIG. 4—Oblong Applicator with frangible Pellet/pouch;

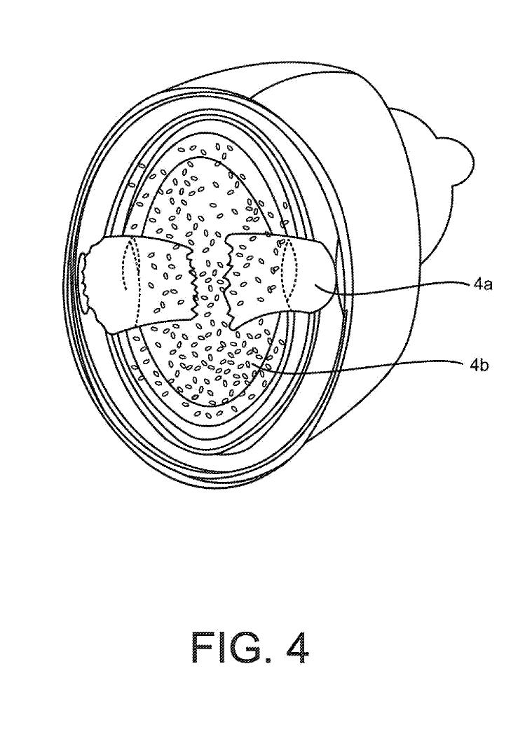

FIG. 5—Penis fracturing pellet/pouch disbursing contents on to penis

FIG. 6—Conventional condom with pellet/pouch applied to interior (male-side) surface

FIG. 7—Anti-Leak condom extensions/grooves molded near to bead-end

FIG. 8—Absorptive element added to inside surface between grooves

FIG. 9—Frangible pellet/pouch attached to foil

FIG. 10—Frangible/squeezeable pellet used to inject treatment

### PREFERRED EMBODIMENTS OF INVENTION

The New Condom System offers a variety of way of treating the male-side of the condom with microbicides/spermicides.

One of the problems with microbicides is that they contribute/cause toxicity in the vagina, along with abrasive changes in vaginal epithelial tissues. These changes potentially weaken the normal blocking power of skin type tissue (even internal lining are a kind of skin) by causing an topical abrasion—resulting in an easier pathway for infection than if the microbicide were not used. Some abrasions have been seen colposcopically during clinical trials.

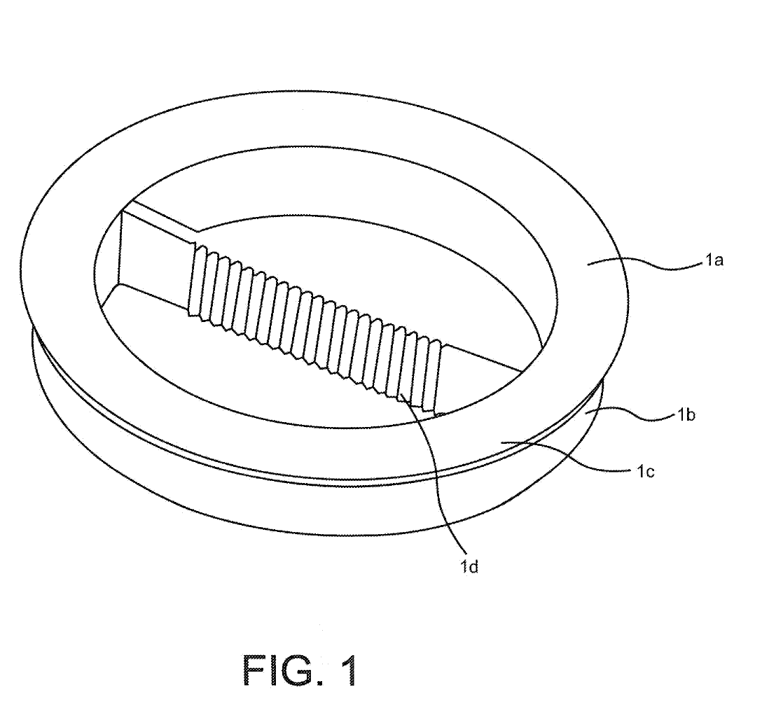

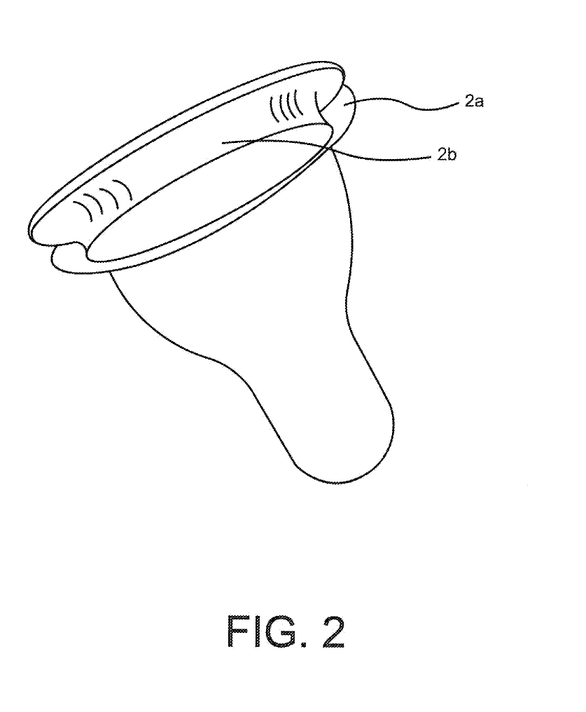

Since treatments applied to the inside of the condom will, over time, migrate by capillary action to the outside of the condom the New Condom System isolates the microbicide to the male-side of the condom. This is accomplished by inserting a detachably-attached plug (see FIG. 1 and FIG. 2) into the bead end of the condom after the condom has been dosed during packaging. The plug consists of a roughly disk-shaped structure(1a) which may or may not be solid or may or may not be a ring (1c) with its center void. At the periphery of the plug a concave groove (1b) is created to hold the finished/bead-end of any rolled condom (2a). The plug is constructed of an elastomer plastic of sufficient rigidity to overcome the compressive forces of the attached condom without deforming. The dimensions of the plug may vary in accordance with the circumference of the condom finished/bead-end with the preferred embodiment being at least 5% greater in circumference than the attached condom so that the condom will hold onto the plug by compressive force. This dimension may be increased which will directly increase the compressive force so as to improve the grip of the condom and resist its dislodging prematurely. Additionally, such additional compressive force will further prevent any treatment introduced into the male-side of the condom from migrating or leaking out during manufacture, shipment, or shelf-storage. The grooved part of the plug is also referred to as its rim(2b). The plug is designed with a finger hold central strut (1d) to permit it to be easily grasped and removed. Additionally, the top most edges of the plug are diminished in two areas further enabling the plug to be easily removed from the condom just prior to donning. The fact that the plug sits in the rolled up section of the condom orients the user as to the correct-way-on donning direction to achieve additional speed in donning as well as to assure that the condom is donned in the correct-way-on orientation so that it may be fully rolled onto the erection.

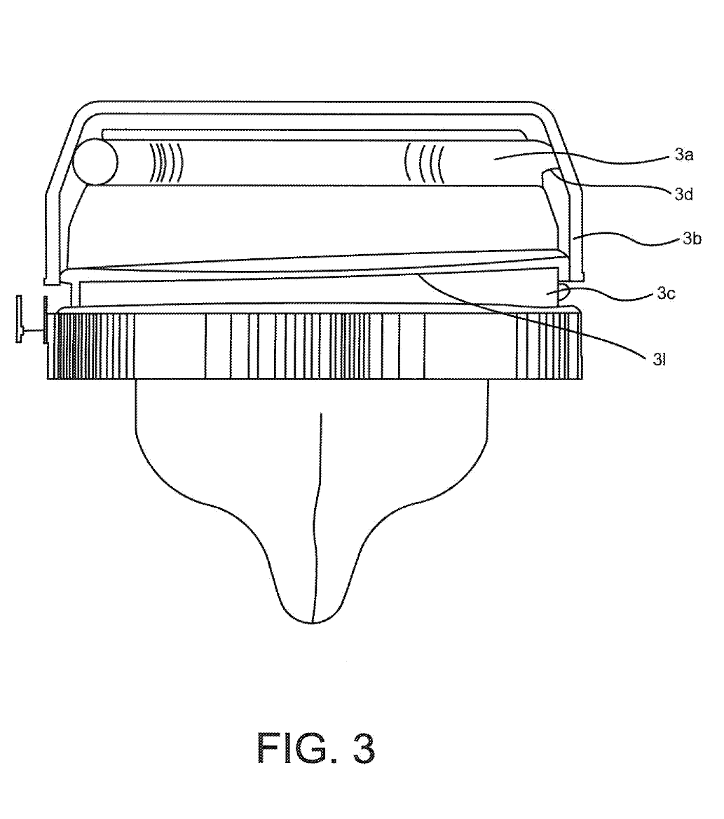

In another preferred iteration the plug's center is removed leaving mainly the grooved rim to which a condom can be attached and held (3a in FIG. 3). In this iteration, the structure of the rim is increased in rigidity to compensate for the loss of its center strut. Such increase may be accomplished by changing the formulation to a more rigid form of plastic ingredient or by increasing the wall thickness of said substrate. The rim is further bifurcated (split into an upper section and a lower section (3b, 3c) to which the condom (see 3d) is actually assembled. The bifurcated halves are further modified with screw threads (see 3e) enabling each half to be mated together with at least ½ turn. Further changes to the top half of the rim causes a partition designed to fit against the outer side of the finished/bead-end of the condom once it is attached in a manner that further compresses (3f) the finished/bead-end so as to provide a further barrier against the migration or leakage of any treatment that was introduced into the male side of the condom after the condom was assembled to rim. The rim may be used as a round applicator to aid in the positioning and donning of the attached condom. Once dosed, the top of the applicator is screwed down over the bottom half (to which the condom has been pre-factory applied) applicator in a manner so that an interior stop on the top cap presses against the side of the condom preventing any of the microbicide from leaking out during shipping or shelf storage (see 3 a-e). This is the same method used to prevent liquids from spilling out from plastic bottles if the bottle were inverted. Prior to use the user unscrews the top half and then uses the bottom half to position and then lower the condom (with its contents held within) over the erection. This action dons the condom on to the penis with the final transfer being accomplished using the edge of the thumb to remove the condom from the applicator. Post application the applicator is discarded.

Another iteration uses a oblong applicator (4) onto which a condom is factory mounted and into which, post such mounting, an amount of microbicide/spermicide is treated. The applicator is similar to the applicator described in Kassman U.S. Pat. No. 7,617,827 except that the top lip of the applicator was modified to better hold a snap cap (not shown) made to fit over and snap down onto a condom that had been applied to the applicator. A snap-on cap is used to hold the microbicide fluid within the condom until the condom is used. The applicator is then used to squeeze open the condom, center it over the erection, and then to lower it over the erection. By thumb rolling the condom off the applicator it is then transferred to the penis and the applicator is discarded.

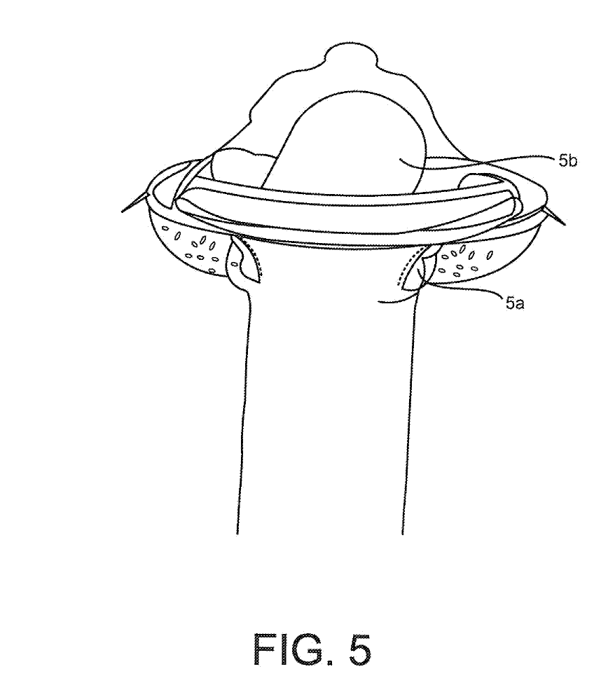

This iteration features a conventional condom configured on an applicator with a frangible pouch/pellet suspended (4a) just above the applicator in such a manner that when the apparatus is lowered onto the erection the penis itself may fracture the pellet (5 seen in FIG. 5). The pouch/pellet is permanently adhered to the applicator (as in the illustration) attached at the lateral sides of the applicator. When the applicator is squeezed open the lateral sides move in opposite direction causing the pre-perforated frangible pouch/pellet to fracture thereby disgorging (4b) the contents that were in the pouch/pellet into the male-side of the condom detachable-attached to the applicator below. The act of squeezing open the applicator in a manner during the moment that the applicator is being used to don the condom causes the pouch/pellet to be fractured (5a) thus releasing its microbicide/spermicide contents onto the penis (5a, 5b). The action of inserting the penis(see FIG. 5) into the condom using the applicator further drives and spreads (5b) the medicinal material from the then fractured pouch toward the business (reservoir-tip) end where it would best available to kill viruses.

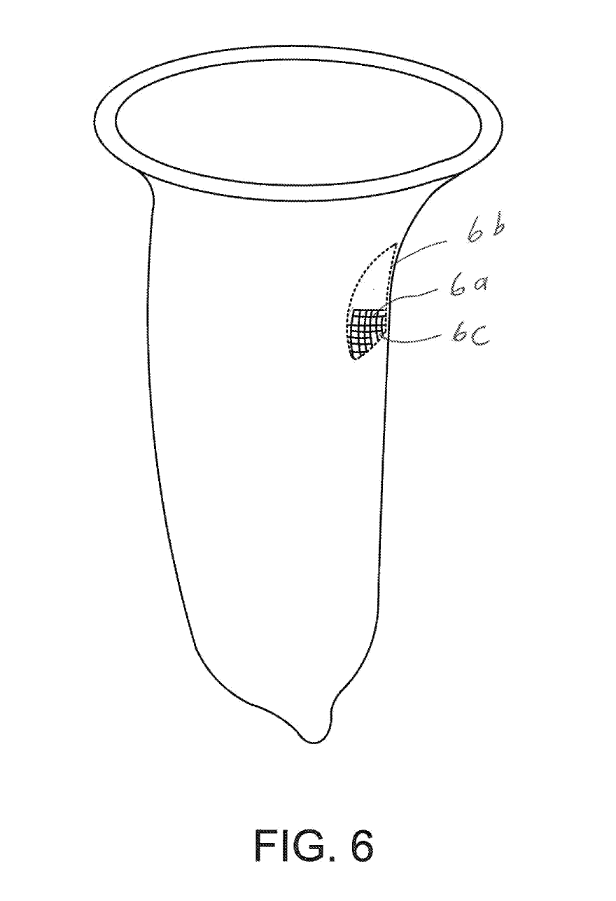

Another iteration positions a frangible pouch/pellet (6a in FIG. 6) tack-attached to the interior wall of the condom (see FIG. 6) and further positions it below the internal rolled-up bead so as to roughly fall on the second two inches of the shaft of an erect penis or to start 2 inches from the point that the pouch/pellet begins measured from the fully unrolled finished/bead-end and extending for 1 additional inch toward the reservoir tip of the condom. The width of the pouch/pellet will be at least ¼ inch and its height above the wall of the condom to which it is attached will be at least ¼ inch. The pouch/pellet will be manufactured of a frangible material compatible with the condom substrate and which may be treated in a manner that will assure that its contents will be disgorged when the penis is inserted and the condom rolled down. The actual fracturing would be caused by rolling it down. To assist in this function the pouch/pellet may be pre-perforated.

Alternatively, the wall of the elastic condom, formed via dipping or heat sealing could be initially formed in a fashion so that a small self-material bladder is formed on the outside of the condom (which becomes the inside of the condom when it is striped off the mandrel. This bladder may or may not be filled with air and it may or may not be totally separated space from the wall of the condom on which it was formed. A secondary step would be needed to fill (see 6b) the bladder with fluid, seal it closed if were not already thus sealed and further create, as necessary, such weak points in the bladder's wall so that it easily ruptured either as the condom, then attached to the applicator were squeezed open or when the male's penis was thrust into the condom during donning.

The actual pouch/pellet would be of a very soft medical grade material (6c)—much softer and more fragile than a condom itself. The material used to hold the pouch/pellet in place would be compatible with the applicator wall to which it becomes applied. A cyanoacrilitic or a rubber cement, or any other material commonly used to hold one type of elastomer to another without damaging the walls of either would be used.

For the iteration where the frangible pouch is actually attached to the wall of the condom, the material used to attach said pouch, and the number and configuration of the points at which such pouch is attached may be different with there being at least one such attached point. Similarly, the number and places as well as the means of attaching a pouch configured to be attached circumferentially to the inside of the condom may vary.

The structure of the frangible pouches may also vary in terms of their shape, the positioning of the frangible elements, the number and shape of the frangible areas or lines of frangibility may also differ. The position of the frangible lines may or may not influence the direction of the flow of the contents of the packages.

The material within any of the frangible pouches may be a spermicide, Or, the material may be a microbicide. Or the material may be a substance which helps the condom better stick to the penis regardless of whether the penis was then wet or dry.

The material within any of the frangible pouches may stimulate the penis to achieve a greater erection. Or, the material may be such to sustain the length of time that the naturally occurring erection is maintained. Or, the material may decrease the sensitivity of the skin of the penis during initial or further friction particularly if the material was time reactive.

Alternatively, in the Condom Applicator model, the frangible pouch (possibly even thin coated paper that was perforated in advance, and then with another very thin coating externally applied post perforation to retain the medicinal product. That pouch could be attached to the sides of the applicator prior to the condom being also attached, so that it positioned itself, depending on the length of the same material loops that would be fitted over the applicator side holding areas, just within the inside of the then being attached condom.

The spreading of fluids is a necessary part of the new condom system. This is different from the initial dispersal. In dispersal, a fluid held within the pellet/pouch would, post rupture of the pouch membrane issue out from the pouch and mostly flow with gravity (therefore, momentarily, at least, toward the bead end of the condom but still on the inner side surface of the condom). This fluid may also be dried and be in the form of an extremely fine powder that would instantly mix with any ambient liquid/lubricant or with pre-ejaculate and with semen. If in liquid form, the flow outside over the open end of the condom, would almost immediately be impeded by the presence of the penis that either immediately post rupture is thrust into the condom as it is donned thru forming a kind of piston pushing the probably still gushing liquid toward the reservoir tip end and/or at least along the inside surface of the condom. Or, should the opening squeezing torque exerted on the applicator and transferred, at least, in part, depending upon the position of the pouch (if glued to the interior condom wall) or on the position of the pouch suspended and hanging freely between the interior sides of the condom and held in place by loops of same pouch material that had been detachably attached to the side indentions formed originally as part of the applicator, were NOT sufficient to fully fracture the frangible pouch, then certainly, the penis itself, as it it thrust into the condom would fracture the pouch membrane and immediately drive the then disgorging liquid or gel toward the reservoir tip end.

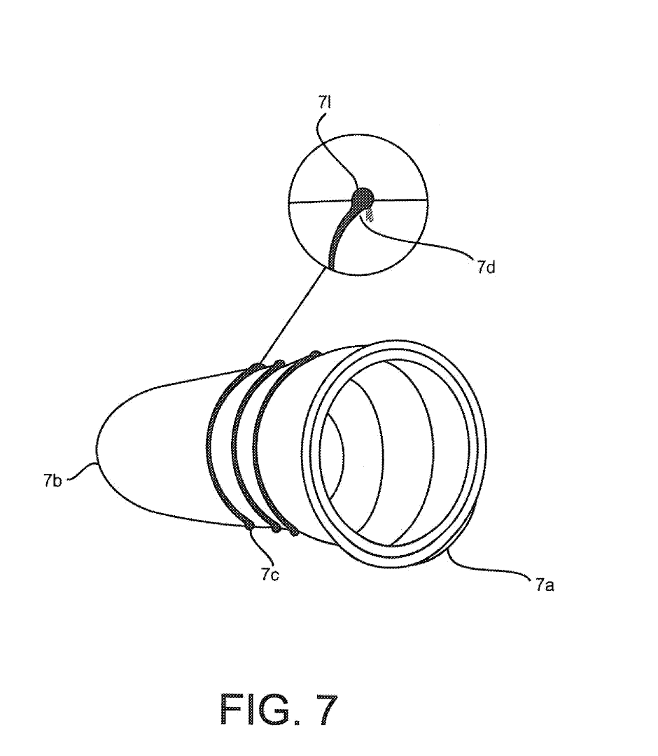

The goal of the above preferred iterations was to introduce a treatment into the male-side of the condom and retain it there. To help accomplish the patent further envisions that certain modification may be required to the condom itself (see FIG. 7). In addition to the iterations above, the bead end (7a) of the condom may be altered. The purpose of this alteration is an additional measure to further retard the migration of fluid treatment that was in the reservoir (7b) end and to further retard leakage of any fluid from the male-side.

A series of at least one internally (toward the penis) facing struts (7c) can be engineered to the dipping mandrel (used in manufacture of the condom, not shown in the drawings). In FIG. 7 a condom is shown with three struts (7c). These circumferential struts, cause by either engraving shaped, specially configured, indentations and/or extensions on mandrel formers, so that when dipped the latex adhered and was formed into one of more circumferential struts. These struts may have single or multiple tiers, they may be rounded or comma shaped (7d), and may be in any configuration that will enable the vulcanized rubber or elastomer substrate, once fully molded to be detached from the former. Additionally, the struts may be of a width and height of at least 1/16 inch and they may be uniformly shaped or have molded extensions to their peripheral configuration to that such extensions may protrude further in parts of the extension than at other parts of the extension. One example of the shape of the extension would be to create a continuous rib or ridge shaped like the aft part of a airplane wing (7e). To allow for penises of different diameters, the thickness (width) of any two such ribs or the amount that they protrude from the condom wall may be different.

Packaging For New Condom System Invention

Conventionally, male condoms are packaged automatically between two layers of foil and/or plastic laminate (which may be layered) and, after they are placed on to the continuous fed substrate, which is crimped and/or pressure/heat sealed, thus sandwiching the inserted condom. During the packaging and prior to sealing an amount of lubricant is added. This is the lubricant that migrates throughout conventional condom packages. The condom itself may or may not be self-oriented into a reservoir-end-up position. This results in confusion by the user as to the correct-way-on direction and often contributes to wrong-way-on donning and damage or tearing of the condom rendering it inefficient as a either a contraceptive, protective, or delivery device for a microbicide or spermicide,

When the user tears open the condom package just prior to use, that user is ordinarily given no sign or signal from the exterior of the package suggesting the correct-way-on for donning, This especially true in low light or dark conditions that ordinarily accompany the condom-use-time continuum. Therefore, when a prospective user who may be rushing to apply the condom, retrieves it from the conventional package, unless he or she looks closely, when the condom immediately is retrieved the condom has a 50% chance of being donned in the wrong direction. When this situation presents itself, if the condom is forced on by pressure it is likely to cover only a portion of the penis and worse, it is likely to tear and to unseat itself during coitus because of intromissive thrusting. Alternatively, the condom is removed, turned around, and rolled on correctly. Or, as is often the case, the act and time of removing it and re-orienting it, the concentration on that effort, and the real time that effort takes, often results in the loss of erection. Another result, one that becomes the most serious, yet very often used, solution is simply for the man to give up entirely on the condom itself and proceed with penetration without it. Of course, all protection is then eliminated in the pursuit of more instant gratification.

The present invention provides a solution for this problem, one which will also permit the selective and, in some iterations, automatic dosing of lubricant on either or both of the internal or external surfaces of the condom. Such dosing can be achieved by simply squeezing open an applicator that was packaged with the condom. Or, it can be achieved by pressure of the penis on a frangible pouch/pellet, as the condom is donned. Or it can be achieved simply by unsnapping a cover cap that had been put in place to cover the condom and its microbicide/spermicide contents. Or it can be achieved by twisting open a different applicator that would then be used to further don the condom accurately.

Regardless of which method, the end result will be a more easily, more speedily applied condom; one which was donned correctly, and one which contained a microbicide that covers the penis (when the condom is fully on) but had not leaked or migrated so as the contact the internal tissues of the female partner.

The present invention envisions three ridges separated from themselves and from the open end of the condom by varying spaces. Each individually and separately designed ridge, actually that will the have the effect of further restricting the return blood flow within the tissue immediately beneath the surface-most layer of the epidermal tissue of the shaft of the penis in that particular area. Their purpose is to constrict blood flow out from the penis resulting in maintenance of the erection.

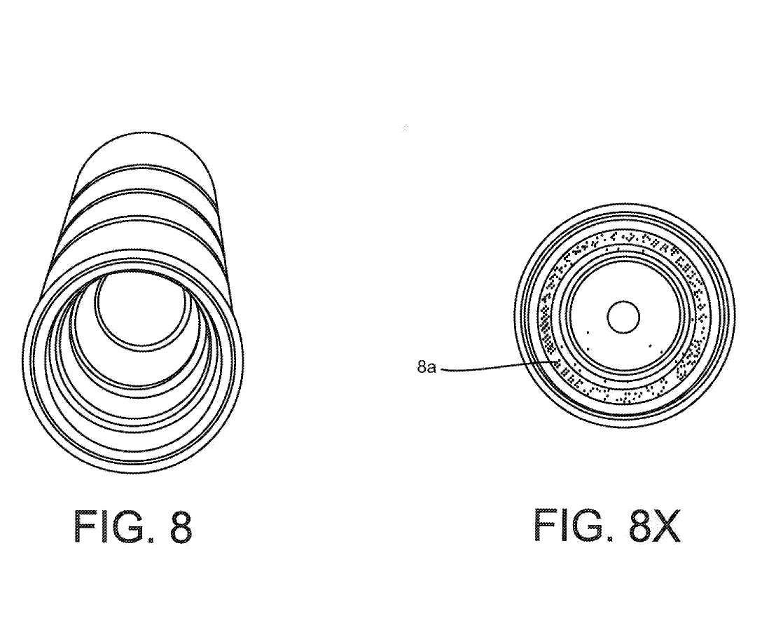

A further embodiment of the present invention combines a thin sponge-like material 8a (FIG. 8) that is oriented and may be applied and secured with latex compatible glue or may be set and held compressively between circumferential ridges on either side of the sponge-like material. The purpose of this material is to act as a further barrier and to absorb the medicinal fluid preventing it from leaking out of the open end of the condom. The sponge-like material would also be able to absorb semen.

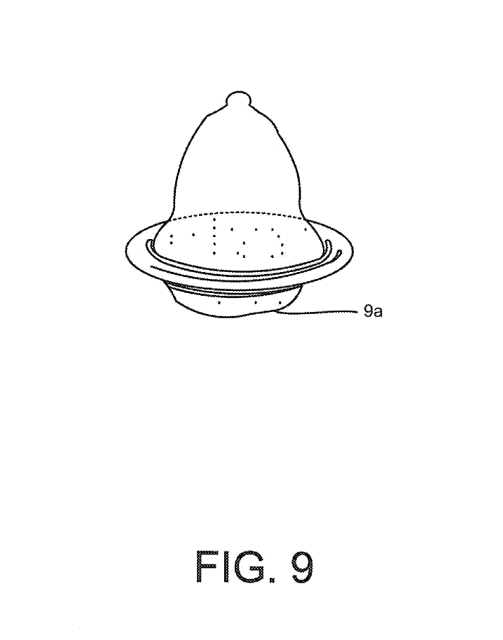

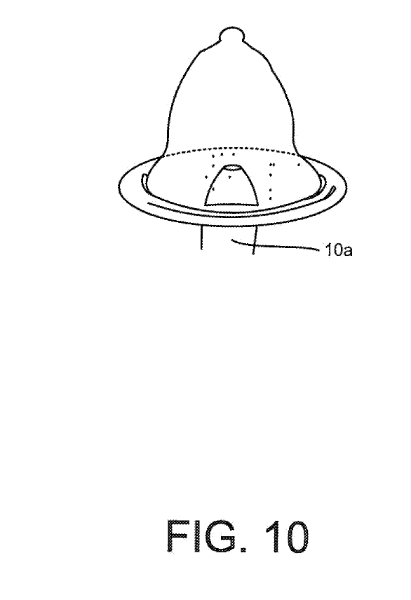

FIGS. 9 and 10 provide illustrations of the means of introducing the medicinal fluid to ordinary conventional condoms or additional lubricant to the embodiments within this patent. FIG. 9 envisions a frangible pouch (9a) that is attached to a foil seal, itself detachable attached to the rolled open end bead of the condom. Said pouch attachment is designed to fracture and disperse its semi-pressurized contents the moment the seal at the open end of the condom is pulled open. Upon such fracture, the contents are then dispersed on the inside of the condom and further pushed to the reservoir tip area by the entry of the erect penis that acts as a piston in a tube. This action simultaneously covers the penis with the medicinal fluid as the condom is further rolled down the shaft.

FIG. 10 shows a similar configuration except that the users shall squeeze a pellet (10a) partially protruding from and held fact to the seal at the open end of the condom. The action of squeezing the frangible pellet fractures its pre-weakened end and sprays its semi-pressurized contents toward the reservoir tip end prior to being pulled from the open end of the condom. The contents are then further propelled toward the closed end and act to cover the penis as the erect penis is inserted into the condom and the balance of the condom is rolled down the shaft of the penis.

Numerous modifications may be made to this invention without departing from its scope as defined in the appended claims.

What is claimed is:

1. A condom comprising:
An elongated body having a distal end that is closed, a proximal end that is opened to apply the condom on a penis, said cylindrical body having including an inner surface and an outer surface; and
a circumferential strut formed radially around said body at a predetermined distance from said proximal end, said strut being sized and shaped to prevent any liquid flow between said inner and outer surfaces while said condom is in use.

2. The condom of claim 1 wherein said strut is disposed at between 1″ and 2-½″ from said proximal end.

3. The condom of claim 1 wherein said body is provided with up to three axially spaced struts.

4. The condom of claim 1 wherein said strut has a cross-sectional shape extending radially of said inner surface.

5. The condom of claim 1 wherein said strut has a generally v-shaped cross section.

6. The condom of claim 1 wherein said strut has the cross-section of an airplane wing,

7. The condom of claim 1 further comprising capsule of medicinal substance positioned to cause said medicinal substance to spread on said inner surface, with said strut preventing said medicinal substance from spreading to said outer surface.

8. A condom comprising:
an elongated body having an inner surface, an outer surface, a distal end closed with a dome shaped tip, and a proximal open end, said elongated body having a generally frustoconical shape with said proximal end having a larger diameter then said distal end; and
a liquid barrier formed on said body, said liquid barrier being sized and shaped to prevent any liquid exchange between inner and outer surfaces.

9. The condom of claim 8 wherein said barrier is sized and shaped to prevent any liquid from flowing from said inner to said outer surface during the use of the condom.

10. A condom assembly comprising:
a cap having a disk shaped body, said disk shaped cap having an outer cylindrical wall with a radial groove, said disk further including an upper surface; and
a condom having a rolled up configuration with a main body portion disposed around outer cylindrical wall and a closed end portion extending across said upper surface.

11. The condom assembly of claim 10 wherein said cap has a bottom portion with a diametrically extending reinforcing member.

12. The condom assembly of claim 10 further comprising a cover attached to said cap to form an enclosure for said condom.
